(19) United States
(12) Patent Application Publication
(10) Pub. No.: US 2022/0233620 A1
(43) Pub. Date: Jul. 28, 2022

(54) METHODS AND RELATED SYSTEMS FOR EXTRACTING ONE OR MORE CHEMICAL COMPOUNDS FROM CANNABIS PLANT MATERIAL

(71) Applicant: 1769474 ALBERTA LTD., Edmonton (CA)

(72) Inventor: Pramodkumar Dinkar JADHAV, Edmonton (CA)

(73) Assignee: 1769474 ALBERTA LTD., Edmonton, AB (CA)

(21) Appl. No.: 17/611,080

(22) PCT Filed: May 13, 2020
(86) PCT No.: PCT/CA2020/050648

§ 371 Date: Nov. 12, 2021

Related U.S. Application Data
(60) Provisional application No. 62/847,681, filed on May 14, 2019.

(51) Int. Cl. A61K 36/185; A61K 31/05; A61K 31/352; B01D 11/02; B01D 15/32; B01D 15/18; B01D 39/06

(52) U.S. Cl. CPC A61K 36/185; A61K 31/05; A61K 31/352; B01D 11/0288; B01D 15/327; B01D 15/18; B01D 2239/0428; B01D 39/06; A61K 2236/37; A61K 2236/53; A61K 2236/55; B01D 2239/0407; B01D 11/0203

(57) ABSTRACT

Methods for extracting one or more chemical compounds from cannabis plant material are provided. In some embodiments, the method may comprise: providing the cannabis plant material; extracting the cannabis plant material with a solvent to produce a solvent extract; and filtering the solvent extract through a filter material to produce a filtered extract. Also provided are related systems. Related cannabis extracts and products comprising cannabis extracts are also provided.

### RELATED APPLICATION

The present application claims priority to U.S. Provisional Patent Application No. 62/847,681, filed May 14, 2019, the entire contents of which are incorporated by reference herein.

### FIELD OF THE DISCLOSURE

The present disclosure relates to methods of processing plant material. More particularly, the present disclosure relates to methods and related systems for extracting one or more chemical compounds from cannabis plant material.

### BACKGROUND

Cannabis refers to plants of the Cannabis genus. The Cannabis genus is generally understood to comprise one species, Cannabis sativa L., although some botanical authorities also recognize Cannabis indica and Cannabis ruderalis. Cannabis plants produce a variety of chemical compounds, including a unique family of terpeno-phenolic compounds called cannabinoids. Cannabinoids may be extracted from cannabis plants and used for a variety of commercial purposes, for example, in pharmaceutical products to treat various medical conditions. Two of the major cannabinoids typically extracted from cannabis plants are cannabidiol (CBD) and Δ⁹-tetrahydrocannabinol (THC).

Solvent extraction and supercritical fluid extraction can be used to obtain cannabinoid-rich extracts from cannabis plant material. However, the resulting extract may require multiple additional processing steps before it is suitable for use in production of a commercial product. For example, a series of additional steps may be performed to decolorize and dewax the extract. Such additional steps can be time-consuming and expensive. In addition, at each step, cannabinoids may be lost, thereby reducing the cannabinoid yield of the final extract.

In addition, for some applications, it may be desirable to reduce the THC content of the cannabinoid-rich extract. For example, as THC may have intoxicating effects in some individuals, it may be desirable to reduce the amount of THC in extracts used in certain pharmaceutical applications. However, removing THC from the extract, without significant loss of CBD and/or other desired cannabinoids, can be difficult.

U.S. patent application Ser. No. 15/981,520 to Shan et al., published as U.S. 2018/0333446, describes a method for extracting cannabinoids from hemp. Hemp refers to varieties of Cannabis sativa L. with low levels of THC, which are typically cultivated for industrial rather than pharmaceutical uses. Shan et al. describes solvent extraction of hemp plant material followed by separation of solids from the solvent extract and distillation of the solvent extract to produce a cannabinoid rich solution. The cannabinoid rich solution is then mixed with a second solvent and subjected to a decolorization and dewaxing process, a THC removal process, or both. The decolorization/dewaxing and THC removal processes each involve passing the second solvent solution through a respective filtration system.

Therefore, methods such as those described by Shan et al. may involve a complex series of steps, and a complex system of physical components, to produce a final extract for use.

### SUMMARY

In one aspect, there is provided a method for extracting one or more chemical compounds from cannabis plant material, the method comprising: providing the cannabis plant material; extracting the cannabis plant material with a solvent to produce a solvent extract; filtering the solvent extract through a filter material to produce a filtered extract; and wherein filtering the solvent extract substantially decolorizes, dewaxes, and reduces the THC content of the solvent extract.

In some embodiments, the one or more chemical compounds comprise one or more cannabinoids.

In some embodiments, the one or more chemical compounds comprise one or more cannabinoids.

In some embodiments, the cannabis plant material has a total cannabinoid content of about 5% or greater.

In some embodiments, the cannabis plant material has a total THC content of about 0.3% or greater.

In some embodiments, filtering the solvent extract reduces the THC content of the solvent extract by between about 50% and about 95% relative to the total THC content of the cannabis plant material.

In some embodiments, the solvent comprises a polar organic solvent.

In some embodiments, the solvent comprises a condensable gas solvent.

In some embodiments, diluting the solvent extract to produce a diluted extract and wherein filtering the solvent extract comprises filtering the diluted extract.

In some embodiments, the filter material comprises a hydrophobic adsorbent.

In some embodiments, the hydrophobic adsorbent comprises activated carbon.

In some embodiments, filtering the solvent extract through the filter material comprises passing the solvent extract through a filtration device retaining the filter material therein.

In some embodiments, the filtration device comprises a radial flow adsorption device.

In some embodiments, the method further comprises concentrating the filtered extract.

In some embodiments, the method further comprises decarboxylating the filtered extract.

In some embodiments, the method further comprises fractionating the solvent extract into two or more solvent extract fractions and wherein filtering the solvent extract comprises filtering at least one of the two or more solvent extract fractions through the filter material.

In some embodiments, the method further comprises fractionating the filtered extract into two or more filtered extract fractions.

In another aspect, there is provided a cannabis extract produced by any embodiments of the methods described herein.

In some embodiments, the cannabis extract has a THC:CBD ratio of about 1:30 to about 1:300.

In some embodiments, the THC:CBD ratio is about 1:100.

In another aspect, there is provided a food product comprising any embodiment of the cannabis extract described herein.

In another aspect, there is provided a beverage product comprising any embodiment of the cannabis extract described herein.

In another aspect, there is provided a cosmetic product comprising any embodiment of the cannabis extract described herein.

In another aspect, there is provided a pharmaceutical product comprising any embodiment of the cannabis extract described herein.

In another aspect, there is provided a veterinary product comprising any embodiment of the cannabis extract described herein.

In another aspect, there is provided a vaporizable product comprising any embodiment of the cannabis extract described herein.

In another aspect, there is provided a system for extracting one or more chemical compounds from cannabis plant material, the system comprising: an extraction device for extracting the cannabis plant material with a solvent to produce a solvent extract; and a filtration device for filtering the solvent extract to produce a filtered extract, the filtration device comprising a radial flow adsorption device.

In some embodiments, the extraction device comprises a chromatography column or a supercritical fluid extraction device.

Other aspects and features of the present disclosure will become apparent, to those ordinarily skilled in the art, upon review of the following description of the specific embodiments of the disclosure.

### BRIEF DESCRIPTION OF THE DRAWINGS

Some aspects of the disclosure will now be described in greater detail with reference to the accompanying drawings. In the drawings:

FIG. 1A is a flowchart of an example method for extracting cannabinoids from a cannabis plant material, according to some embodiments;

FIG. 1B is a flowchart showing additional steps of the method of FIG. 1A;

FIG. 2 is a flowchart of another example method, according to some embodiments;

FIG. 3 is a flowchart of another example method, according to some embodiments;

FIG. 4 is a flowchart of another example method, according to some embodiments;

FIG. 5 is a functional block diagram of an example extraction system, according to some embodiments;

FIG. 6 is a perspective view of an example radial flow adsorption device, according to some embodiments; and

FIG. 7 is a schematic of an example filtration assembly including the radial flow adsorption device of FIG. 6, according to some embodiments.

### DETAILED DESCRIPTION

Generally, the present disclosure provides a method for extracting one or more chemical compounds from cannabis plant material. In some embodiments, the method may comprise: providing the cannabis plant material; extracting the cannabis plant material with a solvent to produce a solvent extract; and filtering the solvent extract through a filter material to produce a filtered extract. In some embodiments, filtering the solvent extract may substantially decolorize, dewax, and reduce the THC content of the solvent extract. Also provided are related systems for extracting one or more chemical compounds from cannabis plant material. Related cannabis extracts and products comprising cannabis extracts are also provided.

As used herein and in the appended claims, the singular forms of “a”, “an, and “the” include plural referents unless the context clearly dictates otherwise.

As used herein, a “chemical compound” may refer to a chemical substance of a specific molecular formula and including any isomers and enantiomers thereof. In some embodiments, one or more of the chemical compounds to be extracted may comprise one or more cannabinoids.

As used herein, “cannabinoid” may refer to any chemical compound capable of acting on a cannabinoid receptor in the human body. In some embodiments, cannabinoid may comprise one or more of cannabidiol (CBD) and Δ⁹-tetrahydrocannabinol (THC). As used herein, “CBD” is intended to include cannabidiolic acid (CBDA) and any possible enantiomer or isomer of CBD or CBDA. As used herein, “THC” is intended to include Δ⁹-tetrahydrocannabinolic acid (THCA) and any possible enantiomer or isomer of THC or THCA.

In some embodiments, cannabinoid may comprise one or more of: cannabinol (CBN), cannabigerol (CBG), cannabigerolic acid (CBGA), (±)-cannabichromene (CBC), (±)-cannabichromenic acid (CBCA), cannabicyclol (CBL), cannabivarin (CBV), tetrahydrocannabivarin (THCV), tetrahydrocannabivarin acid (THCVA), cannabidivarin (CBDV), cannabidivarin acid (CBDVA), cannabigerovarin (CBGV), cannabigerovarin acid (CBGVA), cannabichromevarin (CBCV), any possible enantiomer or isomer thereof, and any other cannabinoid that can be present in cannabis plants.

In some embodiments, one or more of the chemical compounds to be extracted may comprise one or more of terpenes, flavonoids, and any other chemical compound that can be present in a cannabis plant.

As used herein, “cannabis plant” may refer to a plant of the Cannabis genus. The plant of the Cannabis genus may be Cannabis sativa L., Cannabis indica, or Cannabis ruderalis. In some embodiments, the cannabis plant may be a variety or cultivar of industrial hemp. In some jurisdictions, industrial hemp must legally contain less than about 0.2% or 0.3% THC by dry weight of its leaves and flowers. The THC content of industrial hemp is relatively low (i.e. less than about 0.2% or 0.3% THC by dry weight of its leaves and flowers), and its CBD content only somewhat higher, compared to non-hemp, or ‘drug-type’ cannabis varieties or cultivars. In other embodiments, the cannabis plant may be variety or cultivar of cannabis grown for pharmaceutical or other applications (e.g. a “drug-type” variety or cultivar) and may not be a variety or cultivar of industrial hemp.

Within cannabis plants, CBD mainly occurs in the form of cannabidiolic acid (CBDA) and THC mainly occurs in the form of Δ⁹-tetrahydrocannabinolic acid (THCA). CBDA and THCA are converted to CBD and THC, respectively, when cannabis plant material is heated in a process known as decarboxylation, as described in more details below.

In some embodiments, the cannabis plant may have a high CBDA to THCA ratio in any part of the plant, including the leaves and flowers. In some embodiments, the cannabis plant may have a THCA to CBDA ratio of about 1:10 or higher. In some embodiments, the cannabis plant may have a THCA to CBDA ratio of about 1:50 or lower. In some embodiments, the cannabis plant has a THCA to CBDA ratio of between about 1:10 and about 1:50. For example, the cannabis plant may have a ratio of THCA to CBDA of about 1:13 or about 1:23.

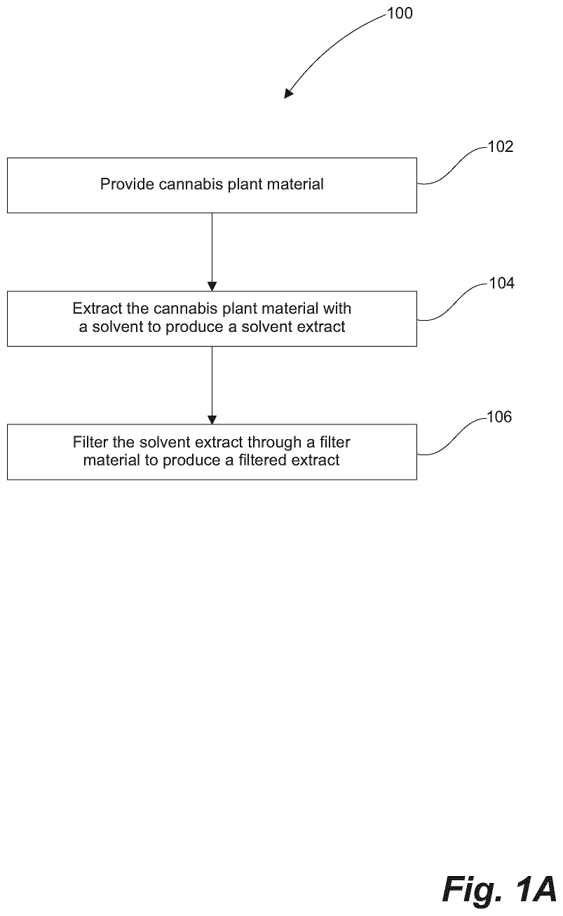

FIG. 1A is a flowchart of an example method 100 for extracting one or more chemical compounds from cannabis plant material, according to some embodiments.

At block 102, the cannabis plant material may be provided. As used herein, “providing” may refer to growing, harvesting, acquiring, buying, or otherwise obtaining the cannabis plant material by any suitable means. In some embodiments, the cannabis plant material may be from one or more cannabis plants. The cannabis plant may be any of the cannabis plants described above.

As used herein, “cannabis plant material” may refer to any substance from one or more cannabis plants. In some embodiments, the cannabis plant material may comprise at least one of floral material, leaves, seeds, stems, stalks, and combinations thereof, from one or more cannabis plants. In some embodiments, the cannabis plant material may comprise floral material from one or more cannabis plants. The floral material may comprise at least one of flowers, flower buds, trichome heads, trichomes, and combinations thereof.

In some embodiments, the cannabis plant material may have high cannabinoid content. In some embodiments, the cannabis plant material may have a total cannabinoid content of about 5% or greater, about 10% or greater, or about 20% or greater by dry weight. In some embodiments, the cannabis plant material may have high THC content and/or high CBD content. In some embodiments, the cannabis plant material may have a total THC content (THC+THCA) about 0.3% or greater, about 1 or greater, or about 5% or greater by dry weight. In some embodiments, the cannabis plant material may have a total THC content of between about 0.3% and about 10% by dry weight. In some embodiments, the cannabis plant material may have a total CBD content (CBD+CBDA) of about 5% or greater, about 10% or greater, or about 20% or greater by dry weight. In some embodiments, the cannabis plant material may have a total CBD content of between about 5% and about 25% by dry weight.

In other embodiments, the cannabis plant material may have low THC content, for example, less than about 0.3%, or less than about 0.2% THC by dry weight. In particular, the THC content of the cannabis plant material will be low in embodiments in which the cannabis plant material is from one or more industrial hemp plants.

In some embodiments, providing the cannabis plant material may comprise providing dried cannabis plant material. The cannabis plant material may be dried using any suitable drying method, including but not limited to, air-drying or drying in a drying tumbler.

In some embodiments, providing the cannabis plant material may comprise providing milled cannabis plant material. The cannabis plant material may be milled using any suitable milling method including dry- or wet-milling methods.

At block 104, the cannabis plant material may be extracted with a solvent to produce a solvent extract. The term “extracted” or “extracting” in this context may refer to contacting the cannabis plant material with a solvent that dissolves at least a portion of the desired chemical compounds of the cannabis plant material, thereby separating the portion of the desired chemical compounds from the remainder of the cannabis plant material. As used herein, “solvent extract” may refer to the substance that has been separated from the remainder of the cannabis plant material and may comprise at least a portion of the desired chemical compounds. In some embodiments, the solvent extract may also comprise at least a portion of remaining solvent and/or at least a portion of unwanted substances. The term “unwanted substance” in this context may refer to any substance other than the desired chemical compounds. Non-limiting examples of unwanted substances include waxes and colorants, as discussed in more detail below.

In some embodiments, the solvent may be polar and/or hydrophilic. Where extraction of CBD is desired, polar solvents may be preferable over non-polar solvents as CBD is more polar than THC. In some embodiments, the solvent may comprise a polar organic solvent such as an alcohol. In some embodiments, the alcohol may comprise at least one of methanol, ethanol, propanol, iso-propanol, butanol, t-butanol, n-butanol, and combinations thereof. In some preferred embodiments, the solvent may comprise ethanol. In some embodiments, the solvent may comprise at least 95% ethanol. As one example, the solvent may comprise anhydrous ethanol with less than about 0.005% water. In other embodiments, the polar solvent may comprise any other suitable polar solvent.

In other embodiments, the solvent may be non-polar and/or hydrophobic. In some embodiments, the solvent may comprise a non-polar organic solvent. In some embodiments, the non-polar organic solvent may comprise at least one of n-heptane, pentane, hexane, acetonitrile, toluene, benzene, chloroform, ethyl acetate, ethyl ether, 1,2-dichloromethane, and combinations thereof. In other embodiments, the non-polar solvent may comprise any other suitable non-polar solvent.

In other embodiments, the solvent may comprise a condensable gas solvent. In some embodiments, the condensable gas solvent may comprise at least one of carbon dioxide (CO₂), a hydrocarbon (e.g. a haloalkane), xenon, nitrous oxide, and sulfur hexafluoride. In some preferred embodiments, the condensable gas solvent may comprise carbon dioxide. In some embodiments, the condensable gas solvent may be in the liquid phase and/or supercritical phase. As used herein, “supercritical phase” or “supercritical fluid” may refer to a fluid state of a condensable gas solvent where it is at or above critical temperature and critical pressure. In some embodiments, the solvent extract produced using a condensable gas solvent may be in the form of a resin or a distillate.

In other embodiments, the solvent may comprise any other suitable solvent, or combination of solvents, that can extract one or more chemical compounds from cannabis plant material.

In some embodiments, extracting the cannabis plant material with the solvent may comprise passing the solvent through the cannabis plant material and recovering the solvent extract. The term “recovering” in this context may refer to collecting, receiving, or otherwise obtaining the solvent extract that has passed through the cannabis plant material. The solvent may be passed through the cannabis plant material by flowing, pumping, or otherwise moving the solvent through the cannabis plant material. Examples of suitable extraction devices are discussed in more detail below.

In some embodiments, the solvent may be passed through the cannabis plant material once. In other embodiments, the solvent may be passed through the cannabis plant material two or more times. For example, the solvent may be passed through the cannabis plant material to produce a first solvent extract and the first solvent extract may then be passed through the cannabis plant material to produce a second solvent extract, and so on until a final solvent extract is recovered. In other embodiments, one or more additional volumes of fresh solvent may be passed through the cannabis plant material and combined with the solvent extract.

In other embodiments, the cannabis plant material may be combined with a suitable volume of solvent for a suitable extraction period. For example, the cannabis plant material may be mixed with the solvent in a suitable vessel and the mixture may be maintained for several minutes up to several hours or days to allow extraction of the one or more chemical compounds. The solvent extract may then be separated from the remaining cannabis plant material. In some embodiments, the solvent extract may be separated from the remaining cannabis plant material by precipitation, pressing, screening, centrifugation, or any other separation suitable method.

In some embodiments, the extraction step at block 104 may be performed at ambient temperature and ambient pressure. In other embodiments, where the solvent comprises a condensable gas solvent, a suitable temperature and pressure may be selected to maintain the solvent in its liquid and/or supercritical phase. In other embodiments, the extraction step may be performed at any other suitable temperature and pressure.

At block 106, the solvent extract may be filtered through a filter material to produce a filtered extract. As used herein, “filtering” may refer to contacting the solvent extract with the filter material so as to remove at least a portion of unwanted substances from the solvent extract. In some embodiments, the filter material may comprise an adsorbent. In some embodiments, at least a portion of unwanted substances may be removed from the solvent extract by adsorption to the adsorbent while the desired chemical compounds may pass through the adsorbent. In other embodiments, the desired chemical compound(s) may adsorb to the adsorbent while unwanted substances may pass through. The desired chemical compound(s) may then be eluted from the adsorbent. In other embodiments, the filter material may remove unwanted substances by any other suitable mechanism.

In some embodiments, the adsorbent may be non-polar and/or hydrophobic. In other embodiments, the adsorbent may be polar and/or hydrophilic. In some embodiments, where a polar/hydrophilic solvent is used at block 104, a non-polar/hydrophobic adsorbent may be used at block 106. In other embodiments, where a non-polar/hydrophobic solvent is used at block 104, a polar/hydrophilic adsorbent may be used at block 106. In other embodiments, any suitable combination of solvent and adsorbent may be used.

In some embodiments, the adsorbent may comprise activated carbon (also known as activated charcoal). While most activated carbons are hydrophobic, embodiments in which the activated carbon may be hydrophilic are also contemplated herein. In some embodiments, the activated carbon may comprise at least one of coal-based activated carbon and wood-based activated carbon. In some embodiments, the activated carbon may comprise at least one of steam-activated and acid-activated activated carbon. In some embodiments, the activated carbon may comprise steam-activated, coal-based activated carbon. In other embodiments, the activated carbon may comprise acid-activated, wood-based activated carbon. In some embodiments, the activated carbon may be in the form of powder, granules, pellets, or any other suitable form of activated carbon. In some embodiments, the activated carbon may further comprise at least one of a binder, a flow aid, and any other suitable additive.

In other embodiments, the adsorbent may comprise silica. In some embodiments, the silica may comprise reverse phase silica such as, for example, reverse phase C18 silica. In other embodiments, the silica may comprise normal phase silica. Reverse phase silica may be hydrophobic, whereas normal phase silica may be hydrophilic.

In other embodiments, the adsorbent may comprise at least one of: clay; alumina; alumina-activated clay; graphite or other carbon-based material; sand; cellulose; synthetic materials including polymers, resins, and gels; diatomaceous earth material; crushed minerals including carbonate, granite, and quartz; zeolites; surface modified natural or synthetic materials; and combinations thereof.

In other embodiments, the filter material may comprise any other suitable filter material or combination of filter materials. In some embodiments, the filter material may comprise a heterogeneous mixture or layered construct of two or more different adsorbents and/or other filter materials.

In some embodiments, filtering the solvent extract may comprise passing the solvent extract through the filter material and recovering the filtered extract. The term “recovering” in this context may refer to collecting, receiving, or otherwise obtaining the filtered extract. In some embodiments, where unwanted substances adsorb to the filter material, the filtered extract may be recovered as the filtrate that has passed through the filter material. Alternatively, where the desired chemical compounds adsorb to the filter material, the filtrate may be discarded and the desired chemical compounds may be eluted from the filter material using any suitable solvent. The solvent extract may be passed through the filter material by flowing, pumping, or otherwise moving the solvent extract through the filter material. Examples of suitable filtration devices are discussed in more detail below.

In other embodiments, the solvent extract may be combined with a suitable amount of the filter material and maintained for a suitable period of time. For example, the solvent extract may be mixed with the filter material in a suitable vessel and maintained for several minutes up to several hours or days. The filtered extract may then be separated from the filter material by precipitation, pressing, screening, centrifugation, or any other suitable separation method.

In some embodiments, filtering the solvent extract though the filter material may substantially decolorize the solvent extract. As used herein, “decolorizing”, (also be referred to as “depigmenting”) may refer to removing colorants present in the solvent extract. The colorants may comprise chlorophylls, phytols, tannins, pigments, and/or any other color-imbuing compounds in the solvent extract. In embodiments in which the filter material comprises activated carbon or another hydrophobic adsorbent, the colorants may be removed from the solvent extract by adsorption to the filter material. A person skilled in the art will understand that although the solvent extract may be substantially decolorized, minor amounts of one or more colorants may remain in the filtered extract. For example, about 10% or less or about 5% or less of the initial colorant content of the solvent extract may remain in the filtered extract.

In some embodiments, filtering the solvent extract though the filter material may substantially dewax the solvent extract. As used herein, “dewaxing” may refer to removing waxes present in the solvent extract. The waxes may comprise terpenes, gums, resins, high molecular weight hydrocarbons, and/or any other wax components of the solvent extract. In embodiments in which the filter material comprises activated carbon or another hydrophobic adsorbent, the waxes may be removed from the solvent extract by adsorption to the filter material. A person skilled in the art will understand that although the solvent extract may be substantially dewaxed, minor amounts of one or more waxes may remain in the filtered extract. For example, about 10% or less or about 5% or less of the initial wax content of the solvent extract may remain in the filtered extract.

In some embodiments, filtering the solvent extract though the filter material may reduce the THC content of the solvent extract. In this context, “reducing the THC content” may refer to removing a portion of the total THC content (THC+THCA) from the solvent extract. In embodiments in which the filter material comprises activated carbon or another hydrophobic adsorbent, THC may be removed from the solvent extract by adsorption to the filter material.

Without being limited by theory, it is believed that since THC is more hydrophobic than CBD, more THC may be adsorbed to a hydrophobic adsorbent filter material (e.g. activated carbon) than CBD, thereby removing a portion of the THC, while allowing the majority of the CBD to pass through the filter material and thereby be present in the filtered extract.

Depending on the other cannabinoids present in the starting cannabis plant material, some other cannabinoids may be at least partially adsorbed to the filter material; while others may largely pass through the filter material and may thereby be present in the filtered extract. For example, CBC and/or CBCA may adsorb to the filter material and CBGA and/or CBDVA may pass through the filter material. Therefore, in some embodiments, filtration of the solvent extract may reduce the content of one or more of THC, CBC, and CBCA and the resulting filtered extract may be enriched for one or more of CBD, CBGA, and CBDVA.

In some embodiments, as the solvent extract is filtered through the filter material, the filter material may become saturated with THC, such as, for example, when the starting cannabis plant material has a relatively high THC content. As a result, once the filter material becomes saturated, no additional THC may be adsorbed, thereby resulting in a larger portion of THC remaining in the filtered extract.

In some embodiments, the portion of THC removed from the solvent extract may be substantially all of the THC that was present in the solvent extract such that there is little to no THC present in the filtered extract. In other embodiments, a portion of THC may still remain in the filtered extract. In some embodiments, the THC content of the filtered extract may be reduced by at least about 10%, 25%, 50%, 75%, 90%, or 95% relative to the THC content of the starting cannabis plant material. In some embodiments, the THC content of the filtered extract may be reduced by between about 10% and about 95%, or between about 50% and about 95%, or between about 75% and about 90%, relative to the THC content of the starting cannabis plant material.

In some embodiments, the filtered extract has a THC:CBD ratio of at least about 1:30, 1:50, 1:100, 1:200, or 1:300. In some embodiments, the cannabis extract has a THC:CBD ratio of between about 1:30 and about 1:300. In some embodiments, the filtered extract has a THC:CBD ratio of between about 1:40 and about 1:164 or between about 1:85 and about 1:290. In some embodiments, the filtered extract has a THC:CBD ratio of about 1:100. A person skilled in the art will understand that, in extracts that have not been decarboxylated (as described in more detail below), the THC and CBD may be primarily in the form of THCA and CBDA.

In some embodiments, the filtered extract may have a relatively high yield of CBD (and/or any other desired chemical compounds). In some embodiments, the CBD content of the filtered extract may be at least about 70%, 80%, or 90% relative to the CBD content of the starting cannabis plant material.

Therefore, in embodiments of the methods described herein, the solvent extract may be filtered only once to substantially dewax, decolorize, and reduce the THC content of the solvent extract. The disclosed methods may thereby reduce or eliminate the need for additional filtration and/or other processing steps such as winterization (i.e. dewaxing). Accordingly, embodiments of the methods described herein may be relatively less expensive and time-consuming than conventional extraction processes with additional processing steps and may produce a filtered extract having a relatively high CBD content (and/or a relatively high content of any other desired chemical compounds) and a relatively low THC content. In some embodiments, the filtered extract may have a relatively high content of CBGA and/or CBDVA and a relatively low content of CBC and/or CBCA.

In addition, the methods described herein may also allow cannabis plant material having relatively high cannabinoid content to be used to produce filtered extracts having relatively low THC content, which may be useful in certain pharmaceutical or veterinary applications and any other applications in which a low THC content is desirable. By using a high cannabinoid starting cannabis plant material, less waste may be produced to obtain a final extract having the same cannabinoid content as that obtained from low cannabinoid plant materials such as industrial hemp.

FIG. 1B is a flowchart showing optional additional steps to method 100 of FIG. 1A, according to some embodiments.

At block 108, the filtered extract may be concentrated to produce a concentrated extract. The term “concentrating” in this context refers to removing at least a portion of the solvent from the filtered extract to increase the concentration of the desired chemical compounds in the concentrated extract. In some embodiments, concentrating the filtered extract may comprise evaporating at least a portion of the solvent. In other embodiments, the filtered extract can be concentrated by any other suitable means.

At block 110, the concentrated extract may be decarboxylated to produce a decarboxylated extract. The term “decarboxylating” or “decarboxylation” in this context refers to treating the extract in a manner to convert at least a portion of THCA to THC and/or CBDA to CBD. In some embodiments, the concentrated extract may be decarboxylated by the application of heat. For example, the concentrated extract may be decarboxylated at approximately 130° C.

In some embodiments, approximately all of the THCA and CBDA is converted to THC and CBD, such that there is little to no THCA and CBDA present in the decarboxylated extract. In other embodiments, minor amounts of THCA and/or CBDA may remain in the decarboxylated extract, for example, less than 15% or less than 10% of the total THC (THC plus THCA) or total CBD (CBD plus CBDA), respectively.

In FIG. 1B, the decarboxylation step at block 110 is shown after the concentration step at block 108; however, in other embodiments, decarboxylation can be performed before the concentration step at block 108 or before the filtration step at block 106 of FIG. 1A. In other embodiments, the concentration step at block 108 can be omitted and the filtered extract can be directly decarboxylated. In embodiments in which a condensable gas solvent (e.g. CO₂) is used as the extraction solvent, the solvent extract may be at least partially decarboxylated during the extraction process (depending on the extraction conditions). In these embodiments, decarboxylation may be concurrent with the extraction step at block 104 and, therefore, a separate decarboxylation step may not be required.

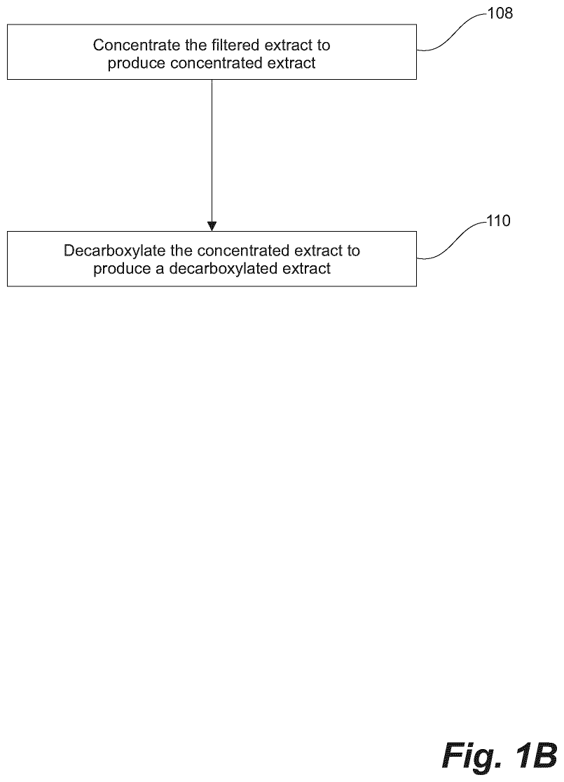

FIG. 2 is a flowchart of another example method 200 for extracting one or more chemical compounds from cannabis plant material, including additional steps, according to some embodiments.

The steps at blocks 202 and 204 may be similar to those at blocks 102 and 104 of method 100 as described above. Briefly, at block 102, the cannabis plant material may be provided. At block 204, the cannabis plant material may be extracted with a solvent to produce a solvent extract.

At block 205, the solvent extract may be diluted with a solvent to produce a diluted extract. As used herein, “diluting” in this context may refer to adding or combining fresh solvent with the solvent extract. In some embodiments, dilution of the solvent extract may increase the total volume of the solvent extract and reduce its viscosity to facilitate the flow of the solvent extract through the filter material at block 206 described below. Hereafter, the solvent used during the extraction step at block 204 may be referred to as the extraction solvent and the solvent used to dilute the solvent extract may be referred to as the dilution solvent.

In some embodiments, the extraction solvent and the dilution solvent may be substantially the same. For example, where ethanol was used as the extraction solvent at block 204, fresh ethanol may be used to dilute the solvent extract at block 205.

In other embodiments, the dilution solvent may be different from the extraction solvent. In some embodiments, where the extraction solvent is a condensable gas solvent, the dilution solvent may be a polar or non-polar organic solvent. For example, where supercritical CO₂ is used as the extraction solvent, an alcohol (e.g. ethanol) may be used to dilute the solvent extract.

In some embodiments, the extraction solvent may be at least partially removed from the solvent extract prior to dilution in the dilution solvent. In some embodiments, the extraction solvent may be at least partially removed by evaporation, boiling, distillation, cyclonic separation, or any other suitable technique. In some embodiments, the solvent may be completely or almost completely removed from the solvent extract and dilution of the solvent extract in the dilution solvent may comprise dissolving the solvent extract in the dilution solvent. This step may be particularly applicable to embodiments in which a condensable gas solvent (e.g. CO₂) is used as the extraction solvent.

At block 206, the diluted extract may be filtered through a filter material to produce a filtered extract. The steps at block 206 may be similar to those at block 106 of method 100 as described above.

In some embodiments, method 200 further comprises one or both of the optional additional steps of FIG. 1B as described above. In some embodiments, the filtered extract is concentrated in a similar manner to that described at block 108. In some embodiments, the filtered extract is decarboxylated in a similar manner to that described at block 110.

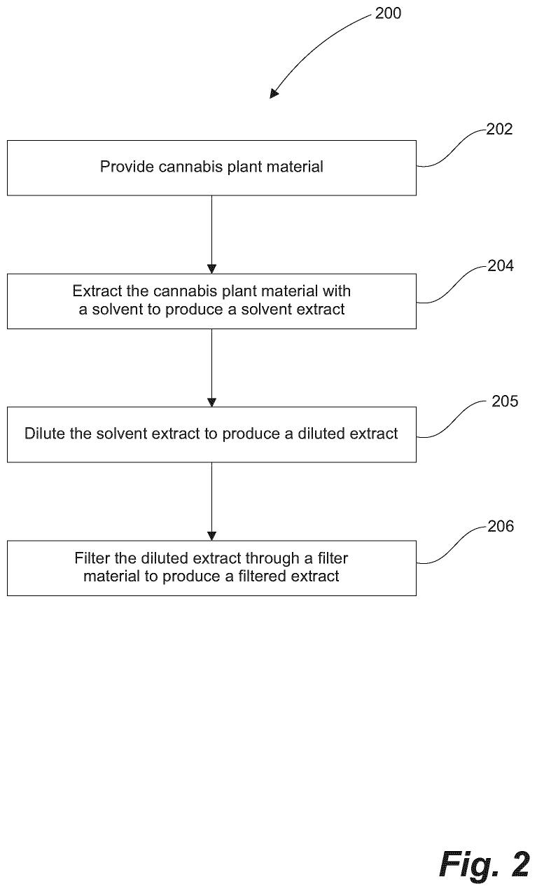

FIG. 3 is a flowchart of another example method 300 for extracting one or more chemical compounds from cannabis plant material, including additional steps, according to some embodiments.

At block 302, the cannabis plant material may be provided. At block 304, the cannabis plant material may be extracted with a solvent to produce a solvent extract. The steps at blocks 302 and 304 may be similar to the steps at block 102 and 104 of method 100 as described above.

At block 305, the solvent extract may be fractionated into two or more solvent extract fractions. The term “fractionating” in this context may refer to recovering the solvent extract in two or more discrete volumes (i.e. “fractions”). In some embodiments, a first volume of solvent can be passed through (or combined with) the cannabis plant material and recovered as a first solvent extract fraction and then a second volume of solvent can be passed through (or combined with) the cannabis plant material and recovered as a second solvent extract fraction, and so on. Alternatively, the solvent can be continuously passed through the cannabis plant material and different solvent extract fractions can be recovered at different times. In some embodiments, the earlier fractions may have higher concentrations of CBD, THC, and/or other desired chemical compounds, while the later fractions may have lower concentrations of CBD, THC, and/or other desired chemical compounds.

It will be understood to a person skilled in the art that although FIG. 3 shows the fractionation steps at block 305 as being performed after the extraction steps at block 304, extraction and fractionation can be performed concurrently.

In some embodiments, each of the solvent extract fractions can be diluted in a solvent to produce respective diluted extract fractions prior to the filtration step at block 306. In some embodiments, at least a portion of the extraction solvent may be removed from the solvent extract prior to dilution, as discussed above with respect to method 200.

At block 306, at least one of the solvent extract fractions may be filtered through a filter material to produce at least one filtered extract fraction. In some embodiments, each of the solvent extract fractions recovered at block 305 may be filtered through the filter material to produce a respective filtered extract fraction. Each fraction may be filtered through the same filter material or through different filter materials.

In some embodiments, method 300 further comprises one or both of the optional additional steps of FIG. 1B as described above. In some embodiments, one or more of the filtered extract fractions are concentrated in a similar manner to that described at block 108. In some embodiments, one or more of the filtered extract fractions are decarboxylated in a similar manner to that described at block 110.

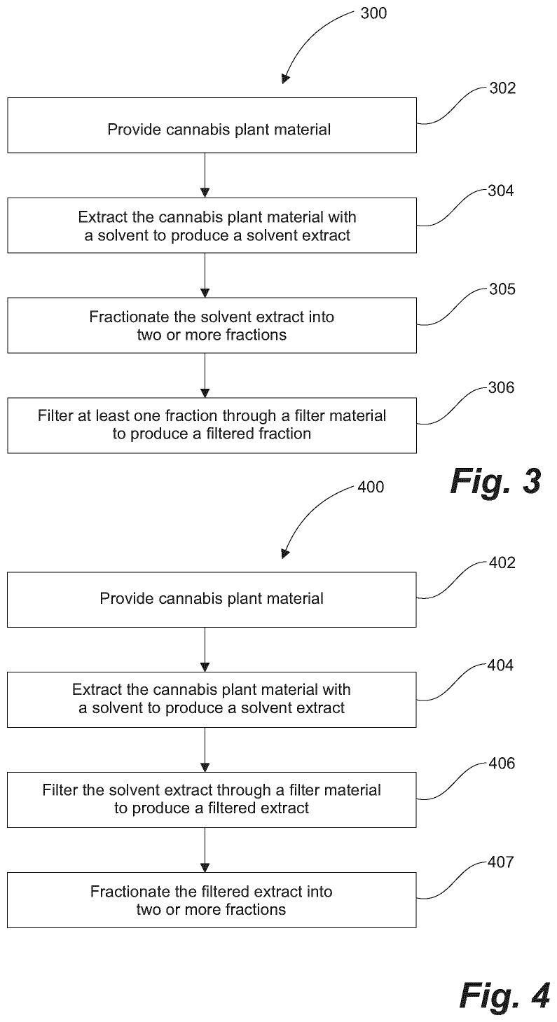

FIG. 4 is a flowchart of another example method 400 for extracting one or more chemical compounds from cannabis plant material, including additional steps, according to some embodiments.

At block 402, the cannabis plant material may be provided. At block 404, the cannabis plant material may be extracted with a solvent to produce a solvent extract. At block 406, the solvent extract may be filtered through a filter material to produce a filtered extract. The steps at blocks 402, 404, and 406 may be similar to the steps at block 102, 104, and 106 of method 100 as described above. In some embodiments, the solvent extract may be diluted as described above at block 205 of method 200.

At block 407, the filtered extract may be fractionated into two or more filtered extract fractions. The filtered extract may be fractionated in a similar manner to the fractionation of the solvent extract as described above at block 305 of method 300.

In some embodiments, the earlier fractions may have a higher CBD content but a lower THC content than the later fractions, as a substantial proportion of the THC may be adsorbed to the filter material. The earlier fractions may thereby have higher CBD to THC ratios than the later fractions. As the filter material becomes saturated with THC, the later fractions may have a higher THC content and thus lower CBD to THC ratios than the earlier fractions. Therefore, in some embodiments, multiple different extracts, with varying CBD to THC ratios, may be produced from the same cannabis plant material.

It will be understood to a person skilled in the art that although FIG. 4 shows the fractionation steps at block 407 as being performed after the filtration steps at block 406, filtration and fractionation can be performed concurrently.

In some embodiments, method 400 further comprises at least one of the optional additional steps of FIG. 1B as described above. In some embodiments, one or more of the filtered extract fractions are concentrated in a similar manner to that described at block 108. In some embodiments, one or more of the filtered extract fractions are decarboxylated in a similar manner to that described at block 110. In some embodiments, the steps of method 400 may be combined with the steps of method 300 as described above such that the solvent extract can be fractionated into two or more solvent extract fractions and each of the filtered fractions can also be fractionated into two or more filtered extract fractions.

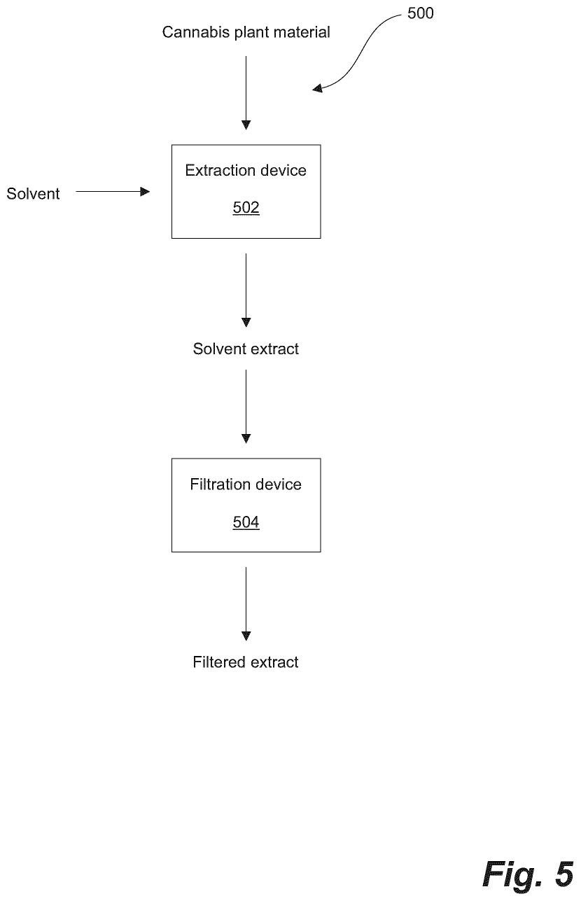

FIG. 5 shows an example extraction system 500 for extracting one or more chemical compounds from cannabis plant material, according to some embodiments. System 500 may implement embodiments of the methods described herein.

Extraction system 500 may comprise an extraction device 502 for extracting the cannabis plant material with a solvent to produce a solvent extract and a filtration device 504 for filtering the solvent extract.

Extraction device 502 may be configured to retain the cannabis plant material therein and receive the solvent to extract the cannabis plant material. In some embodiments, extraction device 502 may comprise an extraction vessel (not shown). In some embodiments, the extraction vessel may comprise an extraction column (not shown). In some embodiments, the extraction column may comprise a chromatography column such as, for example, a glass chromatography column.

In other embodiments, extraction device 502 may comprise a supercritical fluid extraction device (not shown). As used herein, “supercritical fluid extraction device” may refer to any device configured to receive a condensable gas solvent and maintain the condensable gas solvent in a liquid and/or supercritical state. In some embodiments, the supercritical fluid extraction device may be similar to one of the devices disclosed in U.S. patent application Ser. No. 15/841,989, U.S. patent application Ser. No. 15/842,088, Canadian Patent Application No. 2,990,050, and U.S. Pat. No. 10,238,706, incorporated herein by reference.

In other embodiments, extraction device 502 may comprise any other suitable extraction device.

In some embodiments, extraction device 502 may further comprise at least one separator (not shown) to at least partially remove the solvent from the remainder of the solvent extract. For example, the separator may comprise a cyclonic separator. In some embodiments, extraction device 502 may further comprise a mixing device (not shown) to mix the solvent extract with a dilution extract. Alternatively the solvent extract may be manually combined with a dilution extract in any suitable vessel.

In some embodiments, extraction device 502 may be fluidly connected to a solvent reservoir (not shown). In some embodiments, the solvent reservoir may be fluidly connected to extraction device 502 via a first pumping system (not shown) such that the solvent can be pumped through extraction device 502. In other embodiments, extraction device 502 may be configured such that the solvent may be manually added.

Filtration device 504 may be configured to retain a filter material therein and receive the solvent extract to be filtered. The filter material may comprise any suitable filter material, including any of the filter materials described above with respect to the method 100. In some embodiments, filtration device 504 may comprise filtration column (not shown) including, for example, a fixed bed filtration column. In some embodiments, filtration device 504 may comprise a radial flow adsorption device in a suitable housing, as described in more detail below. As used herein, “radial flow adsorption device” may refer to any device in which the filter material is arranged in an approximately annular manner such that fluid flows radially therethrough. One specific example of a radial flow adsorption device is the E-PAK™ radial flow adsorption cartridge and associated housing (Silicycle Inc.™, Quebec City, Quebec, Canada)

In some embodiments, filtration device 504 may be fluidly connected to a second pumping system (not shown) such that the solvent extract can be pumped through filtration device 504. In other embodiments, filtration device 504 may be configured such that the solvent extract can be added manually.

In other embodiments, filtration device 504 may comprise any other suitable filtration device.

In operation, cannabis plant material may be loaded into extraction device 502. The solvent may then be passed through extraction device 502 to produce the solvent extract. In some embodiments, the solvent may be pumped through extraction device 502 via the optional first pumping system. In some embodiments, the solvent may be pumped through extraction device 502 in two or more passes. In some embodiments, the solvent may be pumped from a first end of extraction device 502 to an opposed second end of extraction device 502. Once the solvent reaches the second end of extraction device 502, the flow direction of the solvent may be reversed, and the solvent may be pumped from the second end to the first end. The solvent extract may be recovered from extraction device 502 into any suitable receptacle.

The solvent extract may then be passed through filtration device 504 to produce the filtered extract. In some embodiments, the solvent extract may be pumped via the optional second pumping system through filtration device 504. In some embodiments, prior to pumping the solvent extract through filtration device 504, filtration device 504 may be pre-wetted with a first volume of fresh solvent. The first volume of fresh solvent may be collected as a waste solvent. In some embodiments, a second volume of fresh solvent may then be passed through filtration device 504 and added to the filtered extract. The filtered extract may be recovered from filtration device 504 into any suitable receptacle.

In other embodiments, a fluid line (not shown) may fluidly connect extraction device 502 to filtration device 504, via the second pumping system, thereby allowing the solvent extract from extraction device 502 to be pumped directly into filtration device 504.

In some embodiments, extraction system 500 may further comprise an evaporation device (not shown). The evaporation device may be configured to receive the filtered extract and concentrate the filtered extract by evaporating at least a portion of the remaining solvent in the filtered extract. In some embodiments, the evaporation device may comprise a rotary evaporator. In other embodiments, any other suitable type of evaporation device may be used to concentrate the filtered extract and embodiments are not limited to rotary evaporators. Non-limiting examples of other evaporation devices include a thin film evaporator, a wiped film evaporator, a short-path distillation apparatus, a nitrogen evaporator, or a centrifugal evaporator.

In other embodiments, extraction system 500 may further comprise any other suitable components.

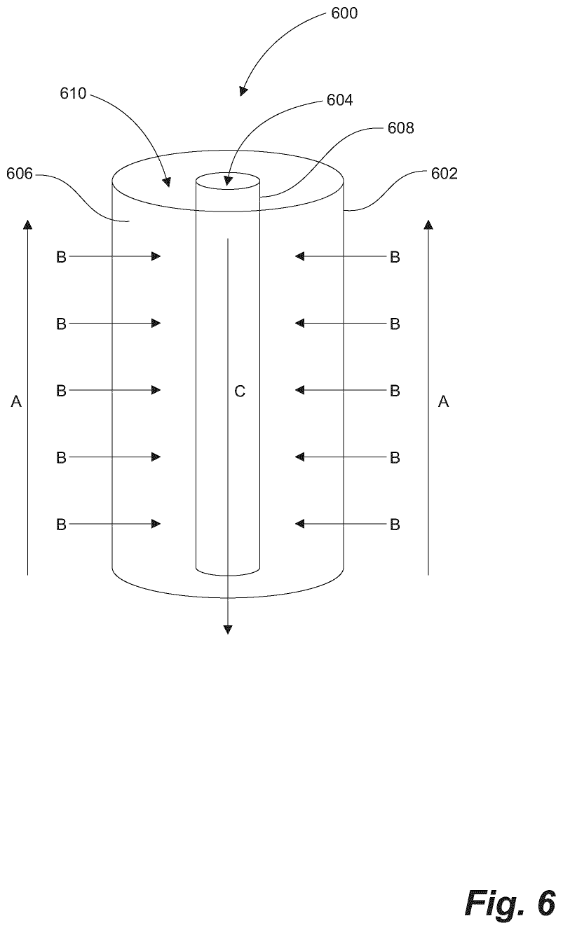

As discussed above, in some embodiments, filtration device 504 may comprise a radial flow adsorption device. An example of a radial flow adsorption device 600 will be described with reference to FIG. 6.

Radial flow adsorption device 600 in this embodiment may comprise a body 602 having a longitudinal conduit 604 therethrough. In some embodiments, body 602 and longitudinal conduit 604 may each be substantially cylindrical. In other embodiments, body 602 and/or longitudinal conduit 604 may be any other suitable shape.

Body 602 may comprise an outer wall 606 and an inner wall 608, inner wall 608 defining longitudinal conduit 604. An annular chamber 610 may be defined between outer wall 606 and inner wall 608. Each of outer wall 606 and inner wall 608 may be comprised of a porous material to allow fluid to pass through body 602 into longitudinal conduit 604. In some embodiments, the porous material may be a mesh material. A filter material such as activated carbon (not shown) may be retained within annular chamber 610, between outer wall 606 and inner wall 608.

Radial flow adsorption device 600 may further comprise a housing (not shown) in which body 602 may be removably mounted. In operation, fluid may be passed into the housing in a longitudinal direction as indicated by arrows A and allowed to flow radially through body 602 as indicated by arrows B towards longitudinal conduit 604. As the fluid passes through body 602 towards longitudinal conduit 604, the fluid may pass through the filter material retained within annular chamber 610, thereby filtering the fluid. The filtered fluid may then flow through longitudinal conduit 604 as indicated by arrow C and out of device 600 such that the filtered fluid may be collected.

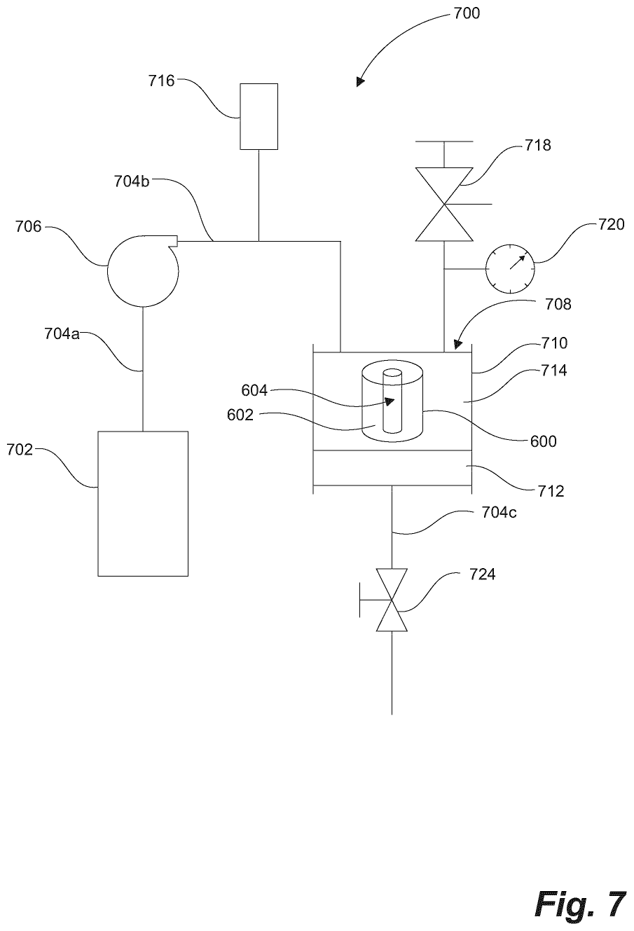

An example of a filtration assembly 700 including radial flow adsorption device 600 of FIG. 6 will be described with reference to FIG. 7. It is to be understood that filtration assembly 700 is shown with very specific details for example purposes only.

Filtration assembly 700 in this embodiment may comprise a feed tank 702, a pump 706, and a filtration column 708.

Feed tank 702 may comprise any suitable vessel in which the solvent extract to be filtered may be held. Feed tank 702 may be fluidly connected to pump 706 via a first fluid line 704a. Pump 706 may be fluidly connected to filtration column 708 via a second fluid line 704b. In some embodiments, pump 706 may comprise a metering pump. In other embodiments, pump 706 may comprise any other suitable type of pump. Pump 706 may be configured to pump the solvent extract from feed tank 702 to filtration column 708 via first and second fluid lines 704a and 704b. In some embodiments, a relief valve 716 may be in fluid communication with second fluid line 704b, between pump 706 and filtration column 708.

Alternatively, in other embodiments, feed tank 702 may be omitted and pump 706 may be directly connected to an extraction device (not shown) via first fluid line 704a. The extraction device may be similar to extraction device 502 of FIG. 5 as described above.

Filtration column 708 may comprise a housing 710 configured to house radial flow adsorption device 600 therein. Housing 710 may comprise a base 712 and an enclosure 714. Base 712 may be configured such that radial flow adsorption device 600 may be mounted thereon. In some embodiments, the longitudinal orientation of radial flow adsorption device 600 may be reversible such that either opposed end of radial flow adsorption device 600 may be mounted on the base 712. Base 712 may comprise an aperture therethrough (not shown) that aligns with longitudinal conduit 604 of radial flow adsorption device 600 when radial flow adsorption device 600 is mounted to base 712. Enclosure 714 may engage base 712 to enclose radial flow adsorption device 600 therein.

In some embodiments, a vent valve 718 may be in fluid communication with filtration column 708 to allow air to be purged from filtration column 708 during operation. In some embodiments, a pressure gauge 720 may be in fluid communication with filtration column 708 to monitor pressure in filtration column 708.

In some embodiments, a metering valve 724 may be in fluid communication with filtration column 708 to control the rate of fluid flow out of filtration column 708. Metering valve 724 may be fluidly connected to filtration column 708 via a third fluid line 704c. Third fluid line 704c may terminate in any suitable receptacle for recovering the filtered extract from filtration column 708.

In operation, radial flow adsorption device 600 may be mounted within housing 710 of filtration column 708. Vent valve 718 may be used to purge air from filtration column 708. Pump 706 may pump the solvent extract from feed tank 702 into filtration column 708. Pressure gauge 720 may be used to monitor pressure in filtration column 708 and relief valve 716 may release fluid to reduce the pressure if needed. Within filtration column 708, the solvent extract may pass through body 602 of radial flow adsorption device 600, exiting through longitudinal conduit 604 and the aperture in base 712 of housing 710 to be received into third fluid line 704c. Metering valve 724 may be adjusted to maintain the pressure and the rate of fluid flow out of filtration column 708 within desired ranges. The filtered extract may be recovered via third fluid line 704c into any suitable receptacle.

In some embodiments, a plurality of radial flow adsorption devices 600 may be mounted in the same filtration column 708. For example, three, seven, or twelve radial flow adsorption devices 600 may be mounted in the same filtration column 708.

Therefore, in some embodiments, decolorizing, dewaxing, and reduction of the THC content of the solvent extract may be performed using a single filtration apparatus.

Also provided in the present disclosure is a cannabis extract. The cannabis extract may be produced by any embodiments of the methods and systems described herein. In some embodiments, the cannabis extract is the filtered extract produced by any embodiment of the methods and systems described herein. In other embodiments, the filtered extract is concentrated and/or decarboxylated (as described elsewhere herein) to produce the cannabis extract. In some embodiments, the concentrated and/or decarboxylated filtered extract is combined with a suitable carrier oil to produce the cannabis extract. In some embodiments, the carrier oil comprises a medium-chain triglyceride (MCT) oil (e.g. coconut oil, vegetable oil, olive oil, etc.).

Also provided herein is a cannabinoid isolate. As used herein, a cannabinoid isolate may refer to a substantially pure preparation of one cannabinoid. For example, the cannabinoid isolate may comprise at least 95%, at least 98%, or at least 99% of one cannabinoid. In some embodiments, the cannabinoid isolate may comprise a CBD isolate. In other embodiments, the cannabinoid isolate may comprise a CBGA or CBDVA isolate. The cannabinoid isolate may be produced from the filtered extract produced by any embodiment of the methods and systems described herein. The cannabinoid isolate may be produced from the filtered extract by one or more of distillation, chromatography, crystallization, and any other suitable method.

Also provided herein is a composition comprising the cannabis extract or the cannabinoid isolate. In some embodiments, the composition may be in the form of a pharmaceutical product, a veterinary product, a natural health product, a dietary supplement, a food product, a beverage product, a cosmetic product, a topical product, and/or a vaporizable product for use with a vaporizer.

In some embodiments, the composition may further comprise at least one pharmaceutically and/or nutritionally acceptable excipient. Non-limiting examples of suitable excipients include fillers, binders, carriers, diluents, stabilizers, lubricants, glidants, coloring agents, flavoring agents, coatings, disintegrants, preservatives, sorbents, sweeteners and any other pharmaceutically or nutritionally acceptable excipient. In other embodiments, the composition may further comprise any other ingredient or combination of ingredients.

Without any limitation to the foregoing, the methods and related cannabis extracts disclosed herein are further described by way of the following examples. However, it is to be understood that these examples are for illustrative purposes only, and should not be used to limit the scope of the present disclosure in any manner.

### Example 1—Radial Flow Adsorption Devices Used in Experiments

Experiments were performed using E-PAK™ radial flow adsorption cartridges (Silicycle Inc., Quebec City, Quebec, Canada). Both lab-scale (5×1 cm and 5×10 cm) and commercial-scale (16.5×50 cm) cartridges were tested.

E-PAK™ cartridges use ECOSORB™ activated carbon sorbents (Graver Technologies LLC, Glasgow, Del., USA). Cartridges with four different types of ECOSORB™ sorbents were used in the experiments as shown in Table 1.

TABLE 1

| Sorbent | Type of Activated Carbon |
|---|---|
| C-941 | Wood-based, acid-activated |
| C-944 | Coal-based, steam-activated |
| C-947 | Wood-based, acid-activated |
| C-948 | Wood-based, acid-activated |

Each E-PAK™ cartridge comprises 60-100 wt % activated carbon (CAS Number 7440-44-0), 25-40 wt % ultrahigh molecular weight polyethylene (CAS Number 9002-88-4), and 0.5-1.5 wt % silica flow aid (CAS Number 7631-86-9). The activated carbon in the cartridges is in the form of powdered activated carbon.

In the lab-scale experiments described below, it was found that the C-944 cartridge was more consistent and reproducible in THCA adsorption than the other three cartridges. Therefore, a commercial-scale C-944 cartridge was utilized for the commercial-scale extraction experiments described below.

### Example 2—Lab-Scale Extraction Experiments (Ethanol Extract)

Dry milled cannabis plant material from Cannabis sativa L. having a starting THCA:CBDA ratio of about 1:13 (about 0.7% THCA and 13% CBDA) was used in the lab-scale experiments. The cannabis plant material was extracted with ethanol to obtain an ethanol extract. Ethanol extracts were then passed through the radial flow adsorption cartridges to obtain filtered extracts. Flow rates for the 5×1 cm cartridges were approximately 5 mL/min. Flow rates for the 5×10 cm cartridges were approximately 30-45 mL/min. The cannabinoid concentrations of filtered extracts were analyzed by ultra-performance liquid chromatography (UPLC).

The results of the lab-scale experiments using 5×1 cm cartridges and 5×10 cm cartridges are shown in Table 2 and Table 3, respectively. Using the 5×1 cm cartridges, THCA:CBDA ratios of 1:30 to 1:85 were observed for the filtered extracts. Using the 5×10 cm cartridges, THCA:CBDA ratios of 1:42 to 1:197 were observed for the filtered extracts.

TABLE 2

| | | | Ethanol | Filtered | CBDA | THCA: | % |
|---|---|---|---|---|---|---|---|
| | | Flow | extract | extract | wt. | CBDA | CBDA |
| | | rate | CBDA | fluid | (g) in | in | yield in |
| | Car- | (mL/ | concentration | volume | filtered | filtered | filtered |
| Run | tridge | min) | (g/L) | (mL) | extract | extract | extract |
| 1 | C944 | 5 | 9.59 | 240 | 2.3 | 1:79 | 74.73 |
| 2 | C944 | 5 | 7.33 | 342 | 2.45 | 1:58 | 58.43 |
| 3 | C944 | 5 | 11.42 | 251 | 1.31 | 1:85 | 42.80 |
| 4 | C941 | 5 | 8.6 | 254 | 2.03 | 1:33 | 65.14 |
| 5 | C947 | 5 | 3.89 | 263 | 1.43 | 1:45 | 44.55 |
| 6 | C948 | 5 | 9.70 | 257 | 2.49 | 1:30 | 56.11 |

TABLE 3

| | | | Ethanol | Filtered | CBDA | THCA: | % |
|---|---|---|---|---|---|---|---|
| | | Flow | extract | extract | wt. | CBDA | CBDA |
| | | rate | CBDA | fluid | (g) in | in | yield in |
| | Car- | (mL/ | concentration | volume | filtered | filtered | filtered |
| Run | tridge | min) | (g/L) | (mL) | extract | extract | extract |
| 7 | C944 | 45 | 12.5 | 1303 | 10.70 | 1:197 | 68.49 |
| 8 | C944 | 30 | 30.25 | 826 | 23.01 | 1:44 | 93.23 |
| 9 | C944 | 30 | 30.5 | 968 | 24.62 | 1:42 | 83.40 |

### Example 3—Commercial-Scale Extraction Experiments (Ethanol Extract)

For the commercial scale experiments, dry milled cannabis plant material from Cannabis sativa L. having a CBDA concentration of approximately 13 wt % and a THCA concentration of about 0.6 wt % was used. Approximately 5-6 kg of cannabis plant material was loaded into a glass chromatography column (BPG 300/500; GE Healthcare Bio-Sciences AB, Uppsala, Sweden) and 40 kg of fresh ethanol was used for extraction. The chromatography column, loaded with the cannabis plant material, was filled from the bottom with ethanol using a pump system at a flow rate of 1.57 mL/minute. Once the ethanol reached the top of the column, the flow was reversed, and the column filled from the top at a flow rate of 0.589 mL/minute. The flow was stopped after approximately 40 kg of ethanol had been flowed through the column. The ethanol extract was collected from the bottom of the column in a carboy.

The radial flow adsorption cartridge (16.5×50 cm) was installed in a cartridge housing. Fresh ethanol (24-32 kg) was passed through the cartridge to wet the cartridge and remove air bubbles. The ethanol was collected as waste ethanol.

A pump system was then assembled to pump the collected ethanol extract through the cartridge. The ethanol extract was pumped through the cartridge at a rate of approximately 2.30 L/minute. The filtered extract was collected in a carboy. A final pass of fresh ethanol (24-32 kg) was then pumped through the cartridge and added to the filtered extract.

The concentrations of the cannabinoids in the filtered extract were determined using UPLC.

The filtered extract was concentrated by evaporating a portion of the remaining ethanol under a rotary evaporator (RTV). The concentrated extract was collected in a carboy.

The results of the commercial-scale experiments using 16.5×50 cm cartridges are shown in Table 4. THCA:CBDA ratios of 1:70 to 1:164 were observed for the filtered extracts.

TABLE 4

| | | | Ethanol | Filtered | CBDA | THCA: | % |
|---|---|---|---|---|---|---|---|
| | | Flow | extract | extract | wt. | CBDA | CBDA |
| | | rate | CBDA | fluid | (g) in | in | yield in |
| | Car- | (mL/ | concentration | volume | filtered | filtered | filtered |
| Run | tridge | min) | (g/L) | (mL) | extract | extract | extract |
| 10 | C944 | 2300 | 24.5 | 78490 | 536.46 | 1:164 | 86.20 |
| 11 | C944 | 2300 | 24.5 | 80690 | 525.36 | 1:99 | 86.40 |
| 12 | C944 | 2300 | 24.5 | 82130 | 468.55 | 1:70 | 71.60 |

### Example 4—Lab-Scale Extraction Experiments (CO₂ Resin)

Dry milled cannabis plant material from Cannabis sativa L. having a starting THCA:CBDA ratio of about 1:13 (about 0.7% THCA and 13% CBDA) was extracted with supercritical carbon dioxide (CO₂) to obtain a cannabis resin (the “pre-decarb CO₂ resin”). The pre-decarb CO₂ resin was decarboxylated at high temperature (>130° C.) to obtain high CBD resin (the “post-decarb resin”). Finally, the post-decarb resin was passed through short path distillation at 160° C. to produce “CBD distillate”. The three cannabis resins: pre-decarb CO₂ resin, post-decarb resin and CBD distillate were used for the experiments as shown in Table 5. In general, each cannabis resin (1.62 g) was dissolved in ethanol (32 mL) to prepare the ethanol extract for runs 1-4. For run 5, 1 g cannabis resin was dissolved in 20 mL ethanol. Ethanol extracts were then passed through the radial flow adsorption cartridges to obtain filtered extracts. Flow rates for the 5×1 cm C944 cartridges were approximately 3 mL/min. The cannabinoid concentrations of filtered extracts were analyzed by ultra-performance liquid chromatography (UPLC).

TABLE 5

| Run | Type of CO₂ Resin |
|---|---|
| 1 | pre-decarb CO₂ resin |
| 2 | pre-decarb CO₂ resin |
| 3 | post-decarb resin |
| 4 | CBD distillate |
| 5 | CBD distillate |

The results of the lab-scale experiments using 5×1 cm cartridges are shown in Table 6. Using the 5×1 cm cartridges, THC:CBD ratios of 1:85 to 1:290 were observed for the filtered extracts.

TABLE 6

| | | | Ethanol | Filtered | CBD | THC: | % |
|---|---|---|---|---|---|---|---|
| | | Flow | extract | extract | wt. | CBD | CBD |
| | | rate | CBD | fluid | (g) in | in | yield in |
| | Car- | (mL/ | concentration | volume | filtered | filtered | filtered |
| Run | tridge | min) | (g/L) | (mL) | extract | extract | extract |
| 1 | C944 | 3 | 33.81 | 126 | 0.84 | 1:100 | 77.78 |
| 2 | C944 | 3 | 33.81 | 135 | 0.72 | 1:96 | 72.30 |
| 3 | C944 | 3 | 28.17 | 196 | 1.03 | 1:87 | 79.23 |
| 4 | C944 | 3 | 28 | 196 | 1.11 | 1:85 | 82.50 |
| 5 | C944 | 3 | 28 | 182 | 0.43 | 1:290 | 76.43 |

It should be apparent to those skilled in the art that more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although particular embodiments have been shown and described, it will be appreciated by those skilled in the art that various changes and modifications might be made without departing from the scope of the invention. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

It is to be understood that a combination of more than one of the approaches described above may be implemented. Embodiments are not limited to any particular one or more of the approaches, methods or apparatuses disclosed herein. One skilled in the art will appreciate that variations, alterations of the embodiments described herein may be made in various implementations without departing from the scope of the claims.

1. A method for extracting one or more chemical compounds from cannabis plant material, the method comprising:
providing the cannabis plant material;
extracting the cannabis plant material with a solvent to produce a solvent extract;
filtering the solvent extract through a filter material to produce a filtered extract; and
wherein filtering the solvent extract substantially decolorizes, dewaxes, and reduces the THC content of the solvent extract.

2. The method of claim 1, wherein the one or more chemical compounds comprise one or more cannabinoids.

3. The method of claim 1, wherein the cannabis plant material has a total cannabinoid content of about 5% or greater and has a total THC content of about 0.3% or greater.

4. (canceled)

5. The method of claim 3, wherein filtering the solvent extract reduces the THC content of the solvent extract by between about 50% and about 95% relative to the total THC content of the cannabis plant material.

6. The method of claim 1, wherein the solvent comprises a polar organic solvent.

7. The method of claim 1, wherein the solvent comprises a condensable gas solvent.

8. The method of claim 7, further comprising diluting the solvent extract to produce a diluted extract and wherein filtering the solvent extract comprises filtering the diluted extract.

9. The method of claim 1, wherein the filter material comprises a hydrophobic adsorbent.

10. The method of claim 9, wherein the hydrophobic adsorbent comprises activated carbon.

11. The method of claim 1, wherein filtering the solvent extract through the filter material comprises passing the solvent extract through a filtration device retaining the filter material therein.

12. The method of claim 11, wherein the filtration device comprises a radial flow adsorption device.

13. The method of claim 1, further comprising concentrating the filtered extract.

14. The method of claim 1, further comprising decarboxylating the filtered extract.

15. The method of claim 1, further comprising fractionating the solvent extract into two or more solvent extract fractions and wherein filtering the solvent extract comprises filtering at least one of the two or more solvent extract fractions through the filter material.

16. The method of claim 1 further comprising fractionating the filtered extract into two or more filtered extract fractions.

17. A cannabis extract produced by the method of claim 1.

18. The cannabis extract of claim 17, wherein the cannabis extract has a THC:CBD ratio of about 1:30 to about 1:300.

19. The cannabis extract of claim 18, wherein the THC:CBD ratio is about 1:100.

20-25. (canceled)

26. A system for extracting one or more chemical compounds from cannabis plant material, the system comprising:
an extraction device for extracting the cannabis plant material with a solvent to produce a solvent extract; and
a filtration device for filtering the solvent extract to produce a filtered extract, the filtration device comprising a radial flow adsorption device.

27. The system of claim 26, wherein the extraction device comprises a chromatography column or a supercritical fluid extraction device.
